(19) United States
(12) Patent Application Publication
(10) Pub. No.: US 2013/0197364 A1
(43) Pub. Date: Aug. 1, 2013

(54) ULTRASOUND DIAGNOSIS APPARATUS HAVING PLURALITY OF DISPLAY UNITS

(71) Applicant: Samsung Electronics Co., Ltd., Gyeonggi-do (KR)

(72) Inventor: Ki-wook HAN, Seoul (KR)

(73) Assignee: SAMSUNG ELECTRONICS CO., LTD., Gyeonggi-do (KR)

(21) Appl. No.: 13/749,862

(22) Filed: Jan. 25, 2013

(30) Foreign Application Priority Data
Jan. 27, 2012 (KR) 10-2012-0008530

(51) Int. Cl. A61B 8/00; A61B 8/08; A61B 8/14

(52) U.S. Cl. CPC A61B 8/462; A61B 8/14; A61B 8/5207; A61B 8/464; A61B 8/54; A61B 8/467
USPC 600/440

(57) ABSTRACT

An ultrasound diagnosis apparatus includes a main body supporting ultrasound equipment that generates ultrasound image signals. First and second display units are electrically coupled to the ultrasound equipment, and display ultrasound images corresponding to the ultrasound image signals. A first coupling unit couples the second display unit and the main body and enables movement of the second display unit relative to the main body. As the position of the second display unit is adjustable, a patient may easily view ultrasound images displayed on the second display unit.

### CLAIM OF PRIORITY

This application claims the benefit of Korean Patent Application No. 10-2012-0008530, filed on Jan. 27, 2012, in the Korean Intellectual Property Office, the disclosure of which is incorporated herein in its entirety by reference.

### BACKGROUND

1. Technical Field

This disclosure relates to ultrasound diagnosis apparatus.

2. Description of the Related Art

An ultrasound signal refers to a sound wave in a high-frequency band higher than an audio frequency (20 Hz through 20 kHz) discernible by humans. An ultrasound diagnosis apparatus delivers an ultrasound signal to tissue or organs in a human body, and obtains an image of the tissue or organs using information derived from the ultrasound signal reflected from the matter within the human body.

Modern ultrasound diagnosis apparatus (hereafter, “ultrasound apparatus,” interchangeably) is small, inexpensive, and capable of displaying an image in real-time. Ultrasound is considered harmless and is thus a preferred diagnosis method; it is also widely used in conjunction with other image diagnosis apparatuses such as an X-ray diagnosis apparatus, a computerized tomography (CT) scanner, a magnetic resonance imaging (MRI) apparatus, a nuclear medical diagnostic apparatus, or the like.

Typical ultrasound apparatus includes a main body which contains ultrasound electronics for generating ultrasound images by processing an ultrasound echo signal received via an ultrasound probe. A display unit for displaying the ultrasound images is mounted on the main body, and a control panel for control of the ultrasound diagnosis apparatus is arranged on a front surface of the main body.

### SUMMARY

The present disclosure provides an ultrasound diagnosis apparatus which has a plurality of display units and facilitates improved manipulation convenience.

According to an exemplary embodiment, an ultrasound diagnosis apparatus includes a main body supporting ultrasound equipment that generates ultrasound image signals. First and second display units are electrically coupled to the ultrasound equipment, and display ultrasound images corresponding to the ultrasound image signals. A first coupling unit couples the second display unit and the main body and enables movement of the second display unit relative to the main body. As a position of the second display unit is adjustable, a patient may easily view ultrasound images displayed on the second display unit. The second display unit may be either a passive display panel or a touchscreen panel for displaying an image and receiving user commands via touch input.

The ultrasound diagnosis apparatus may further include a control panel that is located at a front side of the main body The first coupling unit may couple the second display unit and the control panel so as to allow the second display unit to be movable relative to the control panel. Examples of movement of the second display panel include slidable movement, tilt, and/or left-right rotation.

The first coupling unit may include at least one joint.

The first coupling unit may couple the second display unit to the control panel, whereby the second display unit may be detachable with respect to the control panel, and maintain a displayed image when detached.

The ultrasound diagnosis apparatus may further include a driving unit that supplies a driving force to enable movement of the second display unit.

The ultrasound diagnosis apparatus may further include a memory for storing position information for each user of the second display unit; and a control unit for controlling a position of the second display unit according to the position information stored in the memory, whereby the second display unit may automatically move according to a control by the user.

The ultrasound diagnosis apparatus may further include a second coupling for coupling the control panel and the main body so as to allow the control panel to be movable relative to the main body. The second coupling unit may allow the control panel to tilt or to linearly move with respect to the main body.

The ultrasound diagnosis apparatus may further include a third coupling unit for coupling the first display unit and the main body so as to allow the first display unit to be movable relative to the main body. The third coupling unit may include at least one joint for coupling the first display unit and the main body.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of an ultrasound diagnosis apparatus according to an embodiment of the present invention;

FIG. 2 is a cross-sectional view of a first coupling unit in the ultrasound diagnosis apparatus of FIG. 1;

FIG. 3 illustrates an example of a driving arrangement that may be employed within the ultrasound diagnosis apparatus of FIG. 1;

FIG. 4 is a perspective view of an ultrasound diagnosis apparatus according to another embodiment of the present invention;

FIG. 5 is a perspective view of an ultrasound diagnosis apparatus according to yet another embodiment of the present invention;

FIG. 6 is a perspective view of an ultrasound diagnosis apparatus according to still another embodiment of the present invention;

FIG. 7 is a perspective view of an ultrasound diagnosis apparatus according to a further embodiment of the present invention; and

FIG. 8 is a perspective view of an ultrasound diagnosis apparatus according to yet another embodiment of the present invention.

### DETAILED DESCRIPTION OF EXEMPLARY EMBODIMENTS

Hereinafter, exemplary embodiments of the present invention will be described in detail with reference to the attached drawings. In the drawings, like reference numerals denote like elements, and the thicknesses of layers and regions may be exaggerated for clarity.

As used herein, the term “and/or” includes any and all combinations of one or more of the associated listed items. The word “may” is used to indicate at least an optional element, feature or function of the described embodiment.

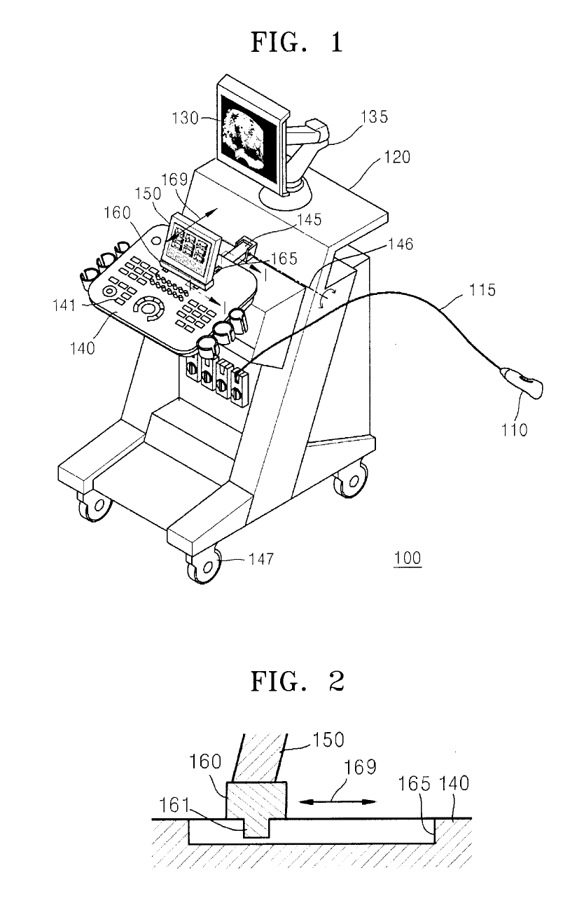

FIG. 1 is a perspective view of an ultrasound diagnosis apparatus 100 according to an embodiment of the present invention. FIG. 2 is a cross-sectional view of a first coupling unit 160, taken along a line I-I of FIG. 1.

Referring to FIG. 1, the ultrasound diagnosis apparatus 100 includes an ultrasound probe 110 for transceiving ultrasound, and a main body 120 supporting or housing ultrasound electronics for generating ultrasound image signals using echo data received from the ultrasound probe 110. A first display unit 130 and a second display unit 150 each display ultrasound images corresponding to the ultrasound image signals. The first coupling unit 160 couples the second display unit 150 to the main body 120 and is designed to allow the second display unit 150 to be movable relative to the main body 120. Examples of the movement of second display unit 150 relative to main body 120 include slidable (e.g. linear) movement, tilting and left-right (i.e., horizontal) rotation.

A control panel 140 may be further arranged on a front side of the main body 120. Here, the front side of the main body 120 denotes the side generally facing the user (i.e., the equipment operator / examiner of the patient) during use of the ultrasound apparatus 100. The second display unit 150 may be coupled to the control panel 140 by the first coupling unit 160 in a location at the front side of the main body 120, and the control panel 140 may be coupled to the main body 120 by a second coupling unit 145. If the control panel 140 is omitted (explained below), the second display unit 150 may be directly coupled to the main body 120 by the first coupling unit 160.

In addition, the first display unit 130 and the main body 120 may be coupled by a third coupling unit 135.

The ultrasound probe 11 is a device that converts a pulse signal into ultrasound, transmits the ultrasound to a target object, and receives an ultrasound echo signal reflected from the target object. A cable that is detachable with respect to the main body 120 may be arranged at an end of the ultrasound probe 110. The ultrasound probe 110 includes a transducer that converts the pulse signal into the ultrasound and converts the reflected ultrasound echo signal into an electrical signal. A signal processing circuit may be included within the ultrasound probe 110, thereby processing electrical signals that are input to or output from the transducer. The transducer may be formed of a plurality of piezoelectric devices that are one-dimensionally or two-dimensionally arrayed.

The ultrasound electronics (interchangeably referred to as “ultrasound equipment”) supported or housed by the main body 120 may include a signal processing unit (not shown) that generates the ultrasound images by using the echo data received from the ultrasound probe 110. (Note that the ultrasound probe 110, cable 115 and control panel 140 are all considered part of the ultrasound equipment supported by the main body 120.) The main body 120 may be embodied in the form of a movable cart mounted on wheels 147.

The first display unit 130 may be arranged on a top portion of the main body 120. The first display unit 130 may display in real-time the ultrasound images obtained from the ultrasound probe 110.

The control panel 140 includes one or more keys or buttons 141 for allowing the user to control operations including selection of an ultrasound image mode, ultrasound strength, signal processing, or the like.

The second display unit 150 may be a display panel that displays a status related to the manipulation of the control panel 140 or signal processing in the ultrasound equipment and also displays the ultrasound images. The second display unit 150 may be embodied as a touchscreen panel configured to receive user commands via touch input. In this case, the user may directly touch and select the ultrasound images or various control menus that are displayed on the touchscreen. That is, when the second display unit 150 is the touchscreen panel, the ultrasound diagnosis apparatus 100 may be controlled by using only the second display unit 150, and in this case, the control panel 140 may optionally be omitted.

The second display unit 150 may perform horizontal slidable movement 169 forward or backward from the front side of the main body 120, by means of the first coupling unit 160. The slidable movement is preferably linear, but curved slot or groove designs are also possible to allow nonlinear sliding.

FIG. 2 illustrates an example of the first coupling unit 160 by which the second display unit 150 is coupled to the control panel 140. A groove 165 that extends in a linear direction is formed in the control panel 140, and a projection part 161 that is inserted into the groove 165 is arranged in an end of the first coupling unit 160. A plurality of the grooves 165 and the projection parts 161 may be formed. The groove 165 functions as a slidable motion guide with respect to the projection part 161. A roller (not shown) or a ball bearing (not shown) may be arranged in the projection part 161 so as to reduce friction or noise that is incurred due to movement of the second display unit 150 therein, and by doing so, the second display unit 150 may move smoothly. The first coupling unit 160 of FIG. 2 is one example of a slidable motion guide; however, alternative designs are also possible to achieve slidable motion in other implementations. Thus, various well-known linear motion guides may be used as the first coupling unit 160.

The first display unit 130 may be a main monitor that displays the ultrasound images obtained from the ultrasound probe 110 whereas the second display unit 150 may be an auxiliary monitor that displays the status related to the manipulation of the control panel 140 or the signal processing in the main body 120.

The second coupling unit 145 coupling the main body 120 and the control panel 140 may have a structure which allows a position or orientation of the control panel 140 with respect to the main body 120 to be varied by the operator. As illustrated in FIG. 1, an example hinge structure of the second coupling unit 145 of FIG. 1 is shown in which the control panel 140 rotates (i.e., the control panel 140 tilts up and down) with respect to a horizontal axis 146. Any suitable hinge structure to enable tilting may alternatively be used. Further, while tilting capability is preferred, the control panel 140 could be provided fixed with respect to the main body 120 in a more simplistic design.

In the exemplary ultrasound diagnosis apparatus 100, a height of the control panel 140 may be adjusted by using the second coupling unit 145 according to a usage environment Distance between the second display unit 150 and the user may be adjusted by the first coupling unit 160. Accordingly, positioning of the second display unit 150 can be optimized by the operator. When the user intends to control operations of the ultrasound diagnosis apparatus 100 using one hand while scanning a patient's body using the ultrasound probe 110 in the other hand, the ability to adjust the second display unit 150 position may significantly improve operational convenience.

The third coupling unit 135 coupling the first display unit 130 to the main body 120 is illustrated with a swivel structure by which a position or orientation the first display unit 130 may be adjusted with respect to the main body 120 so as to improve user convenience. Configurations other than the illustrated swivel may be employed in the alternative. In a simplistic design, the first display unit 130 may be fixed with respect to the main body 120 without provision for physical adjustment.

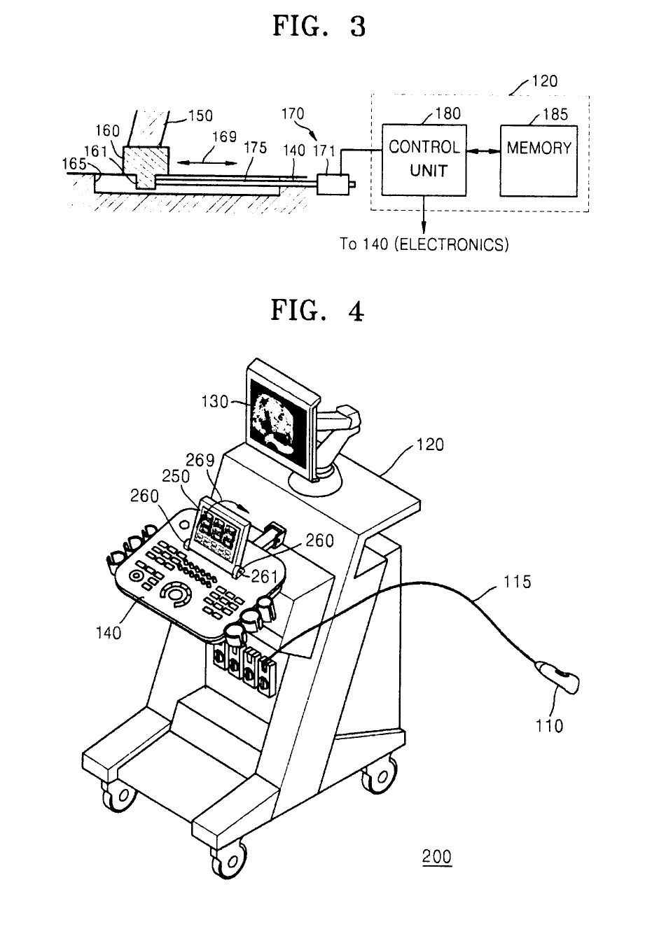

FIG. 3 illustrates an example of a driving arrangement that may be used within the ultrasound diagnosis apparatus 100 of FIG. 1 to cause slidable movement of the second display unit 150. The ultrasound diagnosis apparatus 100 may include a driving unit 170 that supplies a driving force to the first coupling unit 160. The driving unit 170 may include a linear motor 171 and a shaft 175. The linear motor 171 drives the shaft 175 forward or backward in response to a user input command on control panel 140. An end of the shaft 175 is coupled to the projection part 161 of the first coupling unit 160.Movement of the shaft 175 causes the first coupling unit 160 and the second display unit 150 coupled to the first coupling unit 160 to move in a direction controlled by the operator. Other suitable driving arrangements can be employed as alternatives to the illustrated arrangement. If the driving unit 170 is arranged as described above, when a plurality of users use the ultrasound diagnosis apparatus 100 at different times, an optimal position of the second display unit 150 for each user may be stored in a memory 185 in the main body 120. With this design, each user may select his or her own optimal position via suitable commands input on control panel 140, so that a position of the second display unit 150 may be automatically adjusted via the control unit 180. In a more simplistic design, a common adjustment position is stored for all users, which can be updated by a current user. It is noted here that control unit 180 and memory 185 may be either individual components mounted on a printed circuit board contained within main body 120, or realized as portions of already existing control and memory electronics of the ultrasound equipment within main body 120.

FIG. 4 is a perspective view of an ultrasound diagnosis apparatus 200 according to another embodiment of the present invention.

The ultrasound diagnosis apparatus 200 is substantially the same as the previous embodiment 100, except that a first coupling unit 260 of the ultrasound diagnosis apparatus 200 has a coupling structure by which a tilt of a second display unit 250 may be adjusted. That is, the ultrasound diagnosis apparatus 200 includes ultrasound probe 110, main body 120, first display unit 130, control panel 140, and the second display unit 250. The second display unit 250 may be a passive display panel or a touchscreen panel, as described above. The control panel 140 and the second display unit 250 may be coupled to each other by the first coupling unit 260.The control panel 140 and the main body 120 may be coupled to each other by the second coupling unit 145. The first display unit 130 and the main body 120 may be coupled to each other by the third coupling unit 135. The first coupling unit 260 allows the second display unit 250 to be rotated as indicated by legend 269 with respect to a horizontal rotation axis 261, so that a tilt of the second display unit 250 may be adjusted. The tilt adjustment may be done manually, or a suitable drive mechanism (not shown) may be included to enable an electronically controlled tilt via user command on control panel 140. The first coupling unit 260 shown in FIG. 4 is but one example of a coupling structure by which the second display unit 250 can be tilted. Any suitable coupling structure that allows tilt adjustment may be used in the alternative.

In the ultrasound diagnosis apparatus 200 according to the present embodiment, a driving means is not separately arranged in the first coupling unit 260; however, a well-known driving means such as a rotating motor may be used to allow the second display unit 250 to automatically rotate.

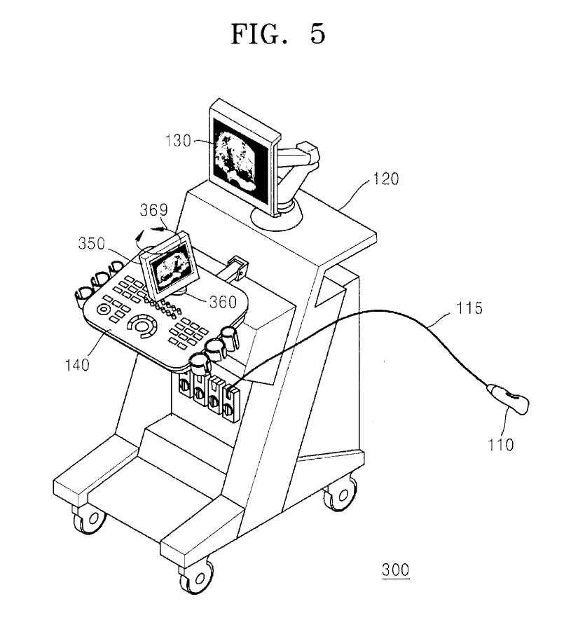

FIG. 5 is a perspective view of an ultrasound diagnosis apparatus 300 according to yet another embodiment of the present invention.

The ultrasound diagnosis apparatus 300 is substantially the same as the previous described embodiments, except that a first coupling unit 360 of the ultrasound diagnosis apparatus 300 has a coupling structure by which a second display unit 350 may rotate in right and left directions. The ultrasound diagnosis apparatus 300 may include ultrasound probe 110, main body 120, first display unit 130, control panel 140, and the second display unit 350. The control panel 140 and the second display unit 350 may be coupled to each other by the first coupling unit 360; he control panel 140 and the main body 120 may be coupled to each other by a second coupling unit 145; and the first display unit 130 and the main body 120 may be coupled to each other by a third coupling unit 135. The first coupling unit 360 may allow rotation 369 of the second display unit 350 in the right and left directions. That is, rotation is enabled in a horizontal plane when the display unit 350 is in a non-tilted state with top and bottom sides oriented horizontally. As described above, the second display unit 350 may be a passive display panel or a touchscreen panel. The first coupling unit 360 shown in FIG. 5 is but one example of a rotatable coupling structure that couples the second display unit 350 to the control panel 140 in such a manner that a display direction of the second display unit 350 may be rotated in a horizontal plane with respect to control panel 140 and main body 120 (e.g., switched between a front direction and a side direction of the control panel 140). However, other rotatable coupling structures may be employed in the alternative.

Because the second display unit 350 may rotate left and right, the second display unit 350 may be used not only as an auxiliary monitor for an examiner situated at the front control panel 140, but also may be used as a monitor viewable by a target subject (i.e., a patient) situated at the side control panel 140. That is, the user performs an ultrasound examination while he or she watches an ultrasound image displayed on the first display unit 130, and at the same time, the user may allow the second display unit 350 to face the target subject by adjusting a position of the second display unit 350, so that the target subject may watch his or her own ultrasound image. Here, the ultrasound image displayed on the second display unit 350 may be a still image that is selected by the examiner, or may be an image that is displayed in real-time. The real-time image may be the same image displayed on the first display unit 130.

In the ultrasound diagnosis apparatus 300 according to the illustrated embodiment, a driving means is not included in the first coupling unit 360; however, in alternative implementations, a well-known driving means such as a rotating motor may be incorporated to allow the second display unit 350 to automatically rotate via user control.

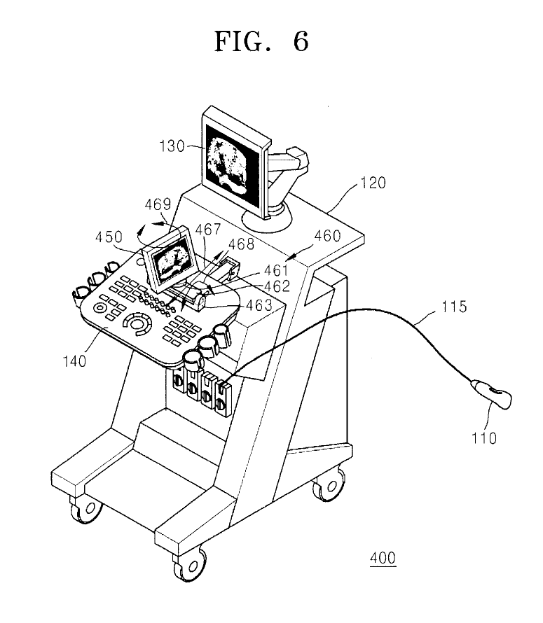

FIG. 6 is a perspective view of an ultrasound diagnosis apparatus 400 according to still another embodiment of the present invention.

The ultrasound diagnosis apparatus 400 is substantially the same as the previous embodiments, except that a first coupling unit 460 of the ultrasound diagnosis apparatus 400 has a coupling structure that allows a second display unit 450 to perform all of slidable (e.g., linear) movement 467, tilting (i.e., vertical plane rotation) 468, and left-right (horizontal plane) rotation 469. Ultrasound diagnosis apparatus 400 may include ultrasound probe 110, main body 120, first display unit 130, control panel 140, and the second display unit 450. The control panel 140 and the second display unit 450 may be coupled to each other by the first coupling unit 460; the control panel 140 and the main body 120 may be coupled to each other by a second coupling unit 145; and the first display unit 130 and the main body 120 may be coupled to each other by a third coupling unit 135. The first coupling unit 460 may be formed as a composite module having a first sub-coupling unit 461 for performing the left-right rotation 469, a second sub-coupling unit 462 for performing the tilting 468, and a third sub-coupling unit 463 for performing the slidable movement 467. The first sub-coupling unit 461 may be positioned on the second sub-coupling unit 462 and perform the left-right rotation 469 with respect to the second sub-coupling unit 462. The second sub-coupling unit 462 may be positioned on the third sub-coupling unit 463 and may tilt 468 with respect to the third sub-coupling unit 463. The third sub-coupling unit 463 may be positioned on the control panel 140 and may perform the slidable movement 467 with respect to the control panel 140, so that a distance from a user may be adjusted. Thus, the second display unit 450, by means of the coupling unit 460, may perform all of the slidable movement 467, the tilting 468, and the left-right rotation 469 with respect to the control panel 140. The first, second, and third sub-coupling units 461, 462, and 463 may correspond to the coupling structures 160, 260, and 360 of the previously described embodiments of FIGS. 1, 4, and 5, respectively.

As described above, the second display unit 450 may be a passive display panel or a touchscreen panel. The first coupling unit 460 shown in FIG. 6 is one example of a coupling structure that allows all of the slidable movement 467, tilting 468, and the left-right rotation 469, but other configurations are possible. Thus, various well-known coupling structures, each of which is capable of composite movements, may be alternatively used as the first coupling unit 460. Furthermore, in the first coupling unit 460, each of the first, second, and third sub-coupling units 461, 462, and 463 has an independent coupling structure, thus, the first coupling unit 460 may have a structure in which any two of the first, second, and third sub-coupling units 461, 462, and 463 are combined.

Since the second display unit 450 may rotate in right and left directions due to the first sub-coupling unit 461, the second display unit 450 may be used as an auxiliary monitor for a user, and by adjusting a position of the second display unit 450 so as to allow the second display unit 450 to face a target subject (i.e., a patient), the second display unit 450 may also be used as a monitor viewable by the patient.

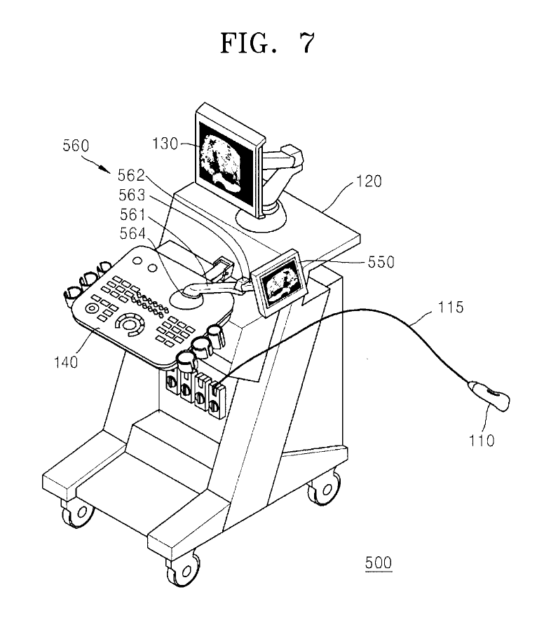

FIG. 7 is a perspective view of an ultrasound diagnosis apparatus 500 according to a further embodiment of the present invention.

The ultrasound diagnosis apparatus 500 is substantially the same as the previous described embodiments, except that a first coupling unit 560 of the ultrasound diagnosis apparatus 500 has a coupling structure by which a second display unit 550 can be moved three dimensionally to a position desired by the operator. Such 3D movement is achievable in the embodiment by means of the first coupling unit 560 configured in the form of a flexible arm structure.

Ultrasound diagnosis apparatus 500 may include ultrasound probe 110, main body 120, first display unit 130, control panel 140, and the second display unit 550. The control panel 140 and the second display unit 550 may be coupled to each other by the first coupling unit 560; the control panel 140 and the main body 120 may be coupled to each other by a second coupling unit 145; and the first display unit 130 and the main body 120 may be coupled to each other by a third coupling unit 135. The first coupling unit 560 may have an arm structure having a first arm 561, a second arm 562, and a joint unit 563 for rotatably coupling the first arm 561 and the second arm 562. Furthermore, the first arm 561 may be coupled to the control panel 140 by a sub-coupling unit 564 so as to be rotatable in right and left directions with respect to the control panel 140. As described above, the second display unit 550 may be a display panel or a touchscreen panel. In the ultrasound diagnosis apparatus 500 of the present embodiment, the first coupling unit 560 has one joint unit 563, but the first coupling unit 560 may have at least two joint units 563.

In the present embodiment, the arm structure of the second display unit 550 is capable of freely moving, so that the second display unit 550 may be used as an auxiliary monitor for a user. By adjusting a position of the second display unit 550 so as to allow the second display unit 550 to face a target subject (i.e., a patient), the second display unit 550 may also be used as a monitor viewable by the patient.

In the illustrated ultrasound diagnosis apparatus 500 according to the present embodiment, a driving means is not separately arranged in the first coupling unit 560; however, in an alternative configuration, a well-known driving means such as a motor or a pressure device may be formed to allow the second display unit 550 to automatically move via user commands.

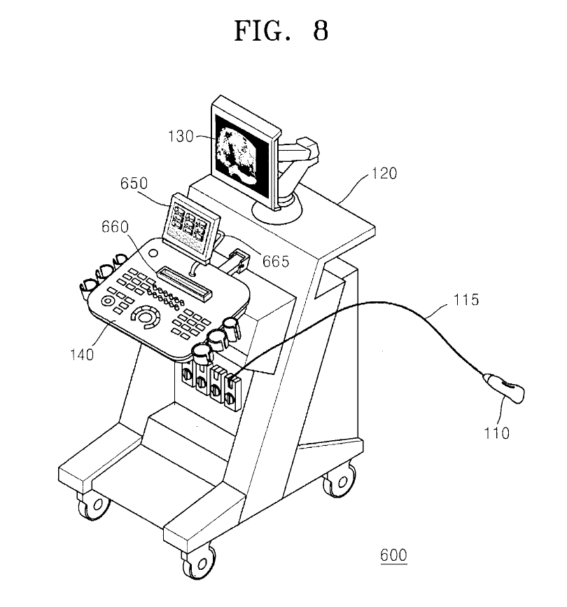

FIG. 8 is a diagram of an ultrasound diagnosis apparatus 600 according to still another embodiment of the present invention.

The ultrasound diagnosis apparatus 600 is substantially the same as the previous embodiments, except that a first coupling unit 660 of the ultrasound diagnosis apparatus 600 has a coupling structure that allows a second display unit 650 to be detachable. That is, referring to FIG. 8, the ultrasound diagnosis apparatus 600 may include ultrasound probe 110, main body 120, first display unit 130, control panel 140, and the second display unit 650. The control panel 140 and the second display unit 650 may be coupled to each other by the first coupling unit 660; the control panel 140 and the main body 120 may be coupled to each other by a second coupling unit 145; and the first display unit 130 and the main body 120 may be coupled to each other by a third coupling unit 135. For example, the first coupling unit 660 may have a groove into which the second display unit 650 is inserted. A cable 665 for electrically connecting the second display unit 650 and the main body 120 may be formed of a flexible material. Thus, the second display unit 650 in a detached state may be used by an examiner or may be used as a patient monitor to be shown to a patient. Second display unit 650 includes battery power to enable the image display to be maintained while detached. The second display unit 650 may also be compositely coupled with one of the coupling structures capable of performing sliding (e.g., linear) movement, tilting, and/or leftward-rightward rotation, as described above with reference to FIGS. 1 through 6. As described above, the second display unit 650 may be a passive display panel or a touchscreen panel.

In any of the above-described embodiments, the second display unit may display the same image as the first display unit 130 in a first operating mode, and may display a different image than that displayed on the first display unit in a second operating mode. The operating modes are selectable by the user via commands input through either the control panel 140 or touch input with the second display unit embodied as a touch screen.

In an ultrasound diagnosis apparatus according to the related art, a display unit such as a lamp indicating a control status, a liquid crystal display (LCD), and the like are formed in a control panel, and in general, the display unit is fixed to the control panel. Recently, the display unit is being replaced with a touchscreen but its structure in terms of being fixed to the control panel is maintained. Thus, generally, when a user intends to manipulate the touchscreen of the control panel while he or she locates an ultrasound probe on a target part of a patient, the user has to move the entire control panel. On the other hand, in the ultrasound diagnosis apparatuses 100, 200, 300, 400, 500, and 600 according to the embodiments of the present invention, the user moves only the second display units 150, 250, 350, 450, 550, and 650 while the control panel 140 remains still and maintains a position or an angle, which is initially set by the user, so that user convenience is improved. In addition, if required during an ultrasound examination, the user may turn the second display units 150, 250, 350, 450, 550, and 650 toward the patient, so that the first display unit 130 may be used as a main monitor that displays ultrasound images to the user in real-time, and the second display units 150, 250, 350, 450, 550, and 650 may be used as an auxiliary monitor that displays selective information (e.g., a selected ultrasound image) to the patient according to selection by the user.

In the ultrasound diagnosis apparatus according to the one or more embodiments of the present invention, the second display unit may be movable relative to the control panel, so that, when an examiner examines a target subject by using a probe, the examiner may adjust a position of the second display unit to an optimal position, and thus manipulation convenience may be improved.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An ultrasound diagnosis apparatus comprising:
a main body supporting ultrasound equipment that generates ultrasound image signals;
a first display unit, electrically coupled to the ultrasound equipment, for displaying ultrasound images corresponding to the ultrasound image signals;
a second display unit, electrically coupled to the ultrasound equipment, for displaying a control status of the main body and for displaying ultrasound images corresponding to the ultrasound image signals; and
a first coupling unit coupling the second display unit and the main body and enabling movement of the second display unit relative to the main body.

2. The ultrasound diagnosis apparatus of claim 1, wherein the second display unit is a touchscreen panel for displaying an image and receiving user commands via touch input.

3. The ultrasound diagnosis apparatus of claim 1, further comprising a control panel located at a front side of the main body and comprises at least one control key.

4. The ultrasound diagnosis apparatus of claim 3, wherein the first coupling unit couples the second display unit and the control panel so as to allow the second display unit to be movable relative to the control panel.

5. The ultrasound diagnosis apparatus of claim 4, wherein
the first coupling unit is configured to allow a display direction of the second display unit to be varied relative to a position of the control panel.

6. The ultrasound diagnosis apparatus of claim 4,
wherein the first coupling unit is configured to allow the second display unit to move linearly, to tilt, or to rotate left and right with respect to the control panel.

7. The ultrasound diagnosis apparatus of claim 4, wherein the first coupling unit comprises at least one joint.

8. The ultrasound diagnosis apparatus of claim 4, wherein the first coupling unit is configured to allow the second display unit to be detachable with respect to the control panel.

9. The ultrasound diagnosis apparatus of claim 3, further comprising a driving unit that supplies a driving force to enable movement of the second display unit.

10. The ultrasound diagnosis apparatus of claim 9, further comprising:
a memory for storing position information for at least one user of the second display unit; and
a control unit for controlling a position of the second display unit according to the position information stored in the memory.

11. The ultrasound diagnosis apparatus of claim 3, further comprising a second coupling unit for coupling the control panel to the main body so as to allow the control panel to be movable relative to the main body.

12. The ultrasound diagnosis apparatus of claim 11, wherein the second coupling unit allows the control panel to tilt or to linearly move with respect to the main body.

13. The ultrasound diagnosis apparatus of claim 11, further comprising a third coupling unit for coupling the first display unit and the main body so as to allow the first display unit to be movable relative to the main body.

14. The ultrasound diagnosis apparatus of claim 13, wherein the third coupling unit comprises at least one joint for coupling the first display unit and the main body.

15. The ultrasound diagnosis apparatus of claim 13, wherein the first display unit is coupled to a top portion of the main body.

16. The ultrasound diagnosis apparatus of claim 4, wherein the first coupling unit is configured to allow the second display unit to all of slide, tilt, and rotate left and right with respect to the control panel.

17. The ultrasound diagnosis apparatus of claim 16, wherein the first coupling unit is in the form of a flexible arm.

18. The ultrasound diagnosis apparatus of claim 17 wherein the flexible arm is composed of a plurality of sub-arms.

19. The ultrasound diagnosis apparatus of claim 1, wherein the second display unit is detachable from the main body and maintains a displayed image when detached.

20. The ultrasound diagnosis apparatus of claim 1, wherein the second display unit displays the same image as the first display unit in a first operating mode, and displays a different image than that displayed on the first display unit in a second operating mode.
